(19) United States
(12) Patent Application Publication
(10) Pub. No.: US 2022/0287844 A1
(43) Pub. Date: Sep. 15, 2022

(54) TRANSFORAMINAL POSTERIOR ATRAUMATIC LUMBAR BIO-IMPLANT

(71) Applicant: LifeNet Health, Virginia Beach, VA (US)

(72) Inventors: Mark Evans, Virginia Beach, VA (US); Gordon Young, Virginia Beach, VA (US); Chris Lundquist, Chesapeake, VA (US); Shane McCauley, Virginia Beach, VA (US)

(73) Assignee: LifeNet Health, Virginia Beach, VA (US)

(21) Appl. No.: 17/636,244

(22) PCT Filed: Aug. 20, 2020
(86) PCT No.: PCT/US20/47106

§ 371 Date: Feb. 17, 2022

Related U.S. Application Data
(60) Provisional application No. 62/889,755, filed on Aug. 21, 2019.

(51) Int. Cl. A61F 2/30; A61F 2/44

(52) U.S. Cl. CPC A61F 2/30771; A61F 2/4465; A61F 2002/30433; A61F 2002/30604; A61F 2002/30784; A61F 2002/30825; A61F 2002/30827; A61F 2002/30904

(57) ABSTRACT

The invention relates to interbody spacers constructed of allograft material. These allograft implants include a first plank of allograft that has a first fusion surface and a first mating surface, opposite the first fusion surface. A second plank of allograft includes second fusion surface and a second mating surface, opposite the second fusion surface. At least one interior plank of allograft has a third mating surface attached to the first mating surface, and a fourth mating surface opposite the third mating surface, attached to the second mating surface. At least one transverse connector interconnects the first, second, and interior planks of the allograft implants. The allograft implants may have at least one transverse passage, and transverse connectors may interconnect the planks through this passage. The ends of transverse connectors may be flush with the fusion surfaces, and those ends may comprise patterned projections.

### CROSS-REFERENCE TO RELATED APPLICATIONS

The present application claims priority to U.S. Provisional Patent Application No. 62/889,755, filed Aug. 21, 2019, entitled “TRANSFORAMINAL POSTERIOR ATRAUMATIC LUMBAR BIO-IMPLANT” the contents of which are incorporated herein by reference in their entirety.

### FIELD

The present disclosure relates generally to interbody fusion implants and more specifically to interbody spacers constructed of allograft material.

### BACKGROUND

Interbody spacers are a type of spinal implant used to treat injuries and degenerative disc conditions in the spine. These implants can be placed in the spine to facilitate fusion after the diseased or damaged disc material is removed.

Interbody spacers can be inserted from different approach angles, such as a posterior approach or transforaminal approach. To insert the spacer, an insertion instrument is attached to one end of the spacer. The instrument is then used to navigate the spacer into the disc space. The end of the spacer that enters the patient first, or “leading end”, is not attached to the instrument. The end of the spacer that enters the patient last, or “trailing end”, is attached to the instrument. Therefore, the surgeon directly controls the trailing end of the spacer, but does not have as much control over the leading end during insertion.

Difficulties can arise during insertion based on the shape of the spacer. Many interbody spacers have a curved or kidney bean shape. A curved or kidney bean shape can be prone to undesired pivot motion and translation during insertion. If pivot motion and translation are not controlled, the spacer can move or slide into an incorrect orientation. Efforts to correct the orientation can result in pushing the spacer beyond the intended implant location. If this occurs, remedial steps are necessary to retrieve the spacer.

Another drawback of conventional spacers is their size requirement. Interbody spacers, particularly in the lumbar region, must have a relatively large height, width and footprint shape. This size requirement makes it challenging to use allograft material. Allograft material is desirable because it promotes fusion between the implant and surrounding bone. Unfortunately, the size of a typical allograft unit is very small due to human bone structure. It is not feasible to produce an interbody spacer having the required size and geometry from a single piece of allograft material. Therefore, many manufacturers choose synthetic materials like polyetheretherketone (PEEK) to produce interbody cages and spacers.

Studies have shown that PEEK can be a safe biomaterial for manufacturing interbody spacers. However, these studies have also shown that the osteoconductive s properties of PEEK are limited. Therefore, spacers formed of PEEK may not promote bone fixation at the implant interface as well as allograft.

### SUMMARY

The drawbacks of conventional interbody spacers are resolved in many respects with interbody spacers according to the present disclosure.

In one aspect of the present disclosure, an allograft implant includes a first plank formed of allograft. The first plank includes a first fusion surface and a first mating surface opposite the first fusion surface. A second plank also formed of allograft includes a second fusion surface and a second mating surface opposite the second fusion surface. At least one interior plank formed of allograft includes a third mating surface attached to the first mating surface of the first plank, and a fourth mating surface opposite the third mating surface and attached to the second mating surface. At least one transverse connector interconnects the first plank, second plank and the at least one interior plank.

In another aspect of the present disclosure, the first plank, second plank and the at least one interior plank define at least one transverse passage. The at least one transverse passage extends through at least a portion of the first plank, at least a portion of the second plank, and at least a portion of the at least one interior plank.

In another aspect of the present disclosure, the at least one transverse passage includes at least one through-passage that extends from the first fusion surface of the first plank to the second fusion surface of the second plank.

In another aspect of the present disclosure, the at least one transverse connector interconnects the first plank, second plank and the at least one interior plank through the at least one transverse passage.

In another aspect of the present disclosure, the at least one transverse connector is press fit through the at least one transverse passage.

In another aspect of the present disclosure, the at least one transverse connector includes a first end and a second end opposite the first end.

In another aspect of the present disclosure, the first end is positioned flush with the first fusion surface of the first plank, and the second end is positioned flush with the second fusion surface of the second plank.

In another aspect of the present disclosure, the first fusion surface includes a first platform and a plurality of first projections raised above the first platform in a first pattern, and the second fusion surface includes a second platform and a plurality of second projections raised above the second platform in a second pattern.

In another aspect of the present disclosure, the first end of the at least one transverse connector comprises a third platform and a plurality of third projections raised above the third platform in a third pattern, and the second end of the at least one transverse connector comprises a fourth platform and a plurality of fourth projections raised above the fourth platform in a fourth pattern.

In another aspect of the present disclosure, the first pattern of first projections blends with the third pattern of third projections to form a first continuous pattern of projections, and the second pattern of second projections blends with the fourth pattern of fourth projections to form a second continuous pattern of projections.

In another aspect of the present disclosure, the first projections and third projections include a first series of longitudinal ridges extending in parallel, and the second projections and fourth projections comprise a second series of longitudinal ridges extending in parallel.

In another aspect of the present disclosure, the first plank and the second plank are arc-shaped.

In another aspect of the present disclosure, a first curved side extends between the first fusion surface and the second fusion surface, and a second curved side extends between the first fusion surface and the second fusion surface opposite the first curved side.

In another aspect of the present disclosure, the first curved side intersects the first platform along a first curved edge, and the second curved side intersects the second platform along a second curved edge.

In another aspect of the present disclosure, the first curved edge and second curved edge are defined by parallel curves.

In another aspect of the present disclosure, the first curved edge, second curved edge, and first series of longitudinal ridges are defined by parallel curves.

In another aspect of the present disclosure, the at least one transverse connector is formed of allograft.

In another aspect of the present disclosure, the at least one transverse connector includes a first transverse pin and a second transverse pin extending parallel to the first transverse pin.

In another aspect of the present disclosure, the at least one transverse connector and the at least one transverse passage are cylindrical.

In another aspect of the present disclosure, a method of manufacturing an allograft implant includes the steps of:

cutting a first plank from allograft material;

cutting a second plank from allograft material;

cutting at least one interior plank from allograft material;

drilling a first transverse passage through the first plank, a second transverse passage through the second plank, and a third transverse passage through the at least one interior plank;

stacking the first plank, second plank and at least one interior plank with the first transverse passage, second transverse passage and third transverse passage coaxially aligned; and

press fitting a transverse connector through the first transverse passage, second transverse passage and third transverse passage to interconnect the first plank, second plank and at least one interior plank.

### BRIEF DESCRIPTION OF THE DRAWING FIGURES

The foregoing summary and the following detailed description will be better understood in conjunction with non-limiting examples shown in the drawing figures, of which:

FIG. 1 is a perspective view of an allograft implant according to one aspect of this disclosure;

FIG. 2 is a front view of the allograft implant of FIG. 1;

FIG. 3 is a cross section view of the allograft implant of FIG. 1 through line A-A;

FIG. 4 is a bottom view of the allograft implant of FIG. 1;

FIG. 5 is a front view of an allograft implant according to another aspect of this disclosure;

FIG. 6 is an exploded view of the allograft implant of FIG. 5; and

FIG. 7 is a block diagram of steps for manufacturing an allograft implant according to another aspect of this disclosure.

### DETAILED DESCRIPTION

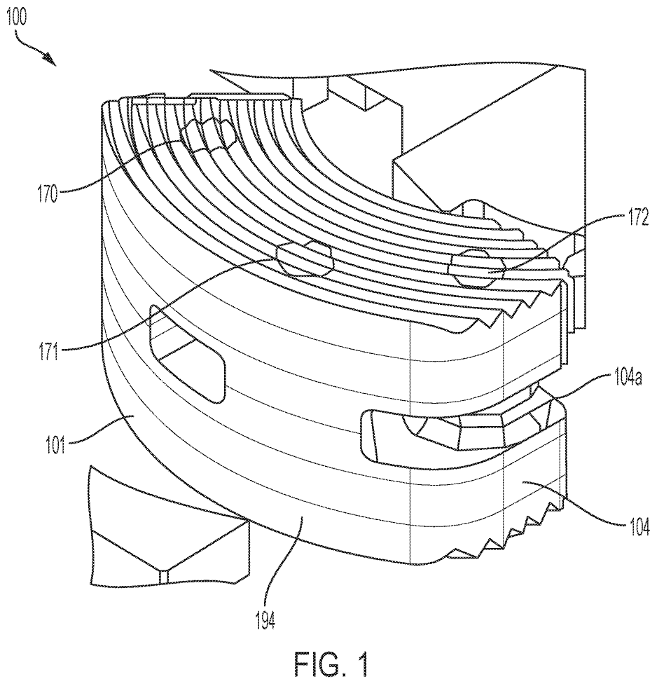

Referring to the drawing figures generally, and FIGS. 1-3 specifically, an interbody spacer or graft 100 is shown according to one example of the present disclosure. Graft 100 is configured for surgical insertion into a vertebral space following disc removal to restore disc height, provide stability, and promote bone fusion. The structure of graft 100 is made up of multiple laminae or planks. The dimensions of each plank are relatively small, making it feasible to manufacture each plank from allograft. The planks are stacked together to collectively form graft 100.

Graft 100 has a kidney bean shaped body 101. Body 101 has a first end 102 that serves as the leading end during insertion. Body 101 also has a second end 104 opposite first end 102. Second end 104 serves as the trailing end during insertion and includes a tool engagement feature 104a for attachment of an insertion tool. The tool engagement feature can have any one of a variety of configurations, the selection of which can depend on the design of the insertion instrument.

First end 102 defines a rounded nose 103. Rounded nose 103 has a first chamfered edge 105 and a second chamfered edge 106. First and second chamfered edges 105, 106 converge toward one another as they extend away from second end 104. This converging geometry forms a tapered leading edge.

Graft 100 includes a first plank 110 formed of allograft. First plank 100 has a first fusion surface 112 and a first mating surface 114 opposite the first fusion surface. Graft 100 also includes a second plank 120 formed of allograft. Second plank 120 has a second fusion surface 122 and a second mating surface 124 opposite the second fusion surface. Body 101 defines a center axis 107 forming an axis of symmetry between first plank 110 and second plank 120.

Grafts according to the present disclosure can consist of two planks only. For example, grafts according to the present disclosure can consist a first plank identical or similar to first plank 110 and a second plank identical or similar to second plank 120 that is connected directly to the first plank. In many cases, however, two planks may not be sufficient to maintain the desired spacing between adjoining vertebrae. Therefore, grafts according to the present disclosure can include an interior section between the first and second planks that provides the additional amount of graft height needed to maintain the desired spacing. Interior sections according the present disclosure can consist of one additional plank, two additional planks, three additional planks, or any other number of planks to provide the additional amount of graft height that is required.

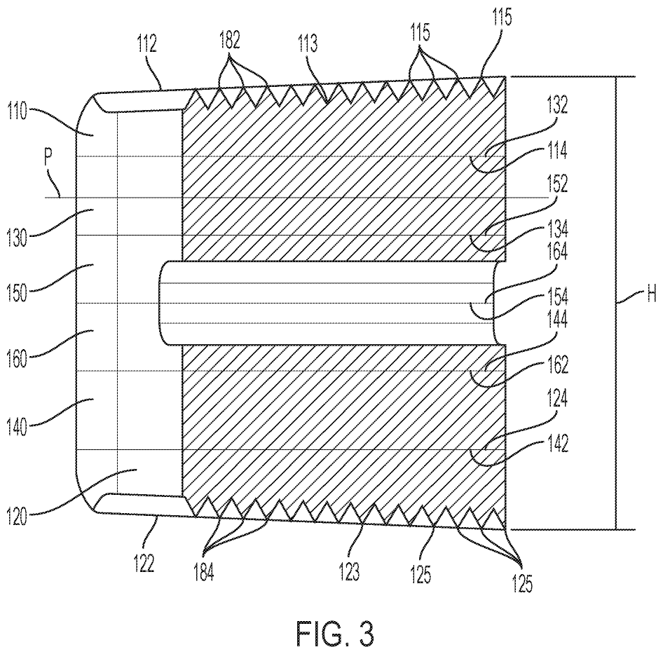

In the present example, graft 100 includes an interior section 108 formed of allograft between first plank 110 and second plank 120. First plank 110, second plank 120 and interior section 108 collectively provide a combined height H, as shown in the side view of FIG. 3. Interior section 108 is made up of four interior planks, including a third plank 130, a fourth plank 140, a fifth plank 150 and a sixth plank 160. Each of planks 110-160 has a generally flat plate shape conforming to a plane P, one of which is shown extending through plank 130. The planes of planks 110-160 extend parallel to one another. Each of planks 110-160 also has a kidney bean shaped profile identical to the kidney bean shape of body 101.

Interior section 108 has a third mating surface 132 defined on third plank 130, and a fourth mating surface 142 on fourth plank 140 that is opposite the third mating surface. Third mating surface 132 is attached to first mating surface 114 on first plank 110. Fourth mating surface 142 is attached to second mating surface 124 on second plank 120.

The planks making up interior section 108 have mating surfaces that interconnect as well. Third plank 130 has a fifth mating surface 134 that is attached to a sixth mating surface 152 on fifth plank 150. Fourth plank 140 has a seventh mating surface 144 that is attached to an eighth mating surface 162 on sixth plank 160. Finally, fifth plank 150 has a ninth mating surface 154 attached to a tenth mating surface 164 on sixth plank 160.

Grafts according to the present disclosure can include one or more connectors to hold the planks together in a vertically stacked arrangement. For example, grafts according to the present disclosure can have one or more elongated connectors that extend transversely to the planes of the planks. Transverse connectors can extend through some of the planks or all of the planks to connect them together. In a six plank design, for example, a first transverse connector can extend through the first, second, third and fourth planks, and a second transverse connector can extend through the third, fourth, fifth and sixth planks. In another example, first and second transverse connectors can extend through all six planks. Transverse connectors can extend parallel to one another and perpendicular to the planes of the planks. Alternatively, transverse connectors can extend in a non-parallel manner at angles that are not perpendicular to the planes of the planks.

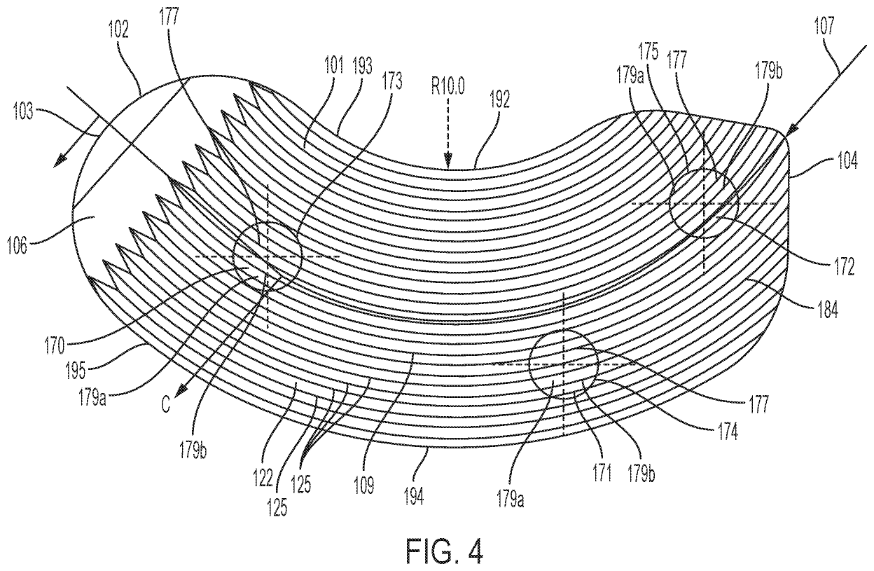

Referring to FIG. 4, graft 100 includes three transverse connectors in the form of transverse pins. A first transverse pin 170 extends through all of the planks 110-160 at a first location near first end 102 of body 101. A second transverse pin 171 extends through all of the planks 110-160 at a second location near a midportion 109 zs of body 101. A third transverse pin 172 extends through all of the planks 110-160 at a third location near second end 104 of body 101.

Planks 110-160 define transverse passages adapted to receive transverse pins 170-172 in a press fit. Each of planks 110-160 is formed with a section of each passage that aligns with sections defined in the other planks to form continuous passages through body 101. Thus, body 101 defines a first transverse passage 173 that receives first transverse pin 170, a second transverse passage 174 that receives second transverse pin 171, and a third transverse passage 175 that receives third transverse pin 172. In this arrangement, transverse pins 170-172 extend through all of the planks 110-160.

First, second and third transverse passages 173-175 each begin at first fusion surface 112 on first plank 110 and end at second fusion surface 122 on second plank 120. Therefore, each of transverse passages 173-175 constitutes a through-passage that extends through body 101 in its entirety. It will be appreciated that the transverse passages need not be through-passages, but can extend through only a portion of the body, as suggested earlier. For example, a first transverse passage may only extend through the first, second, third, and fourth planks, and a second transverse passage may only extend through the third, fourth, fifth and sixth planks. Therefore, grafts according to the present disclosure can feature passages having one end that terminates inside the graft, and/or passages that are through-passages that terminate at fusion surfaces on the exterior of the body.

Referring back to FIG. 3, first fusion surface 112 has a first platform 113 and a plurality of first projections 115 raised above the first platform in a first pattern. Second fusion surface 122 includes a second platform 123 and a plurality of second projections 125 raised above the second platform in a second pattern. First and second projections 115, 125 follow a curvature and have two functions. The first function of projections 115, 125 is to engage cancellous bone material and fuse with the adjoining vertebrae. The second function of projections 115, 125 is to guide graft 100 along a curved path as the graft is inserted into the disc space so that the graft is guided to its final position in the proper orientation.

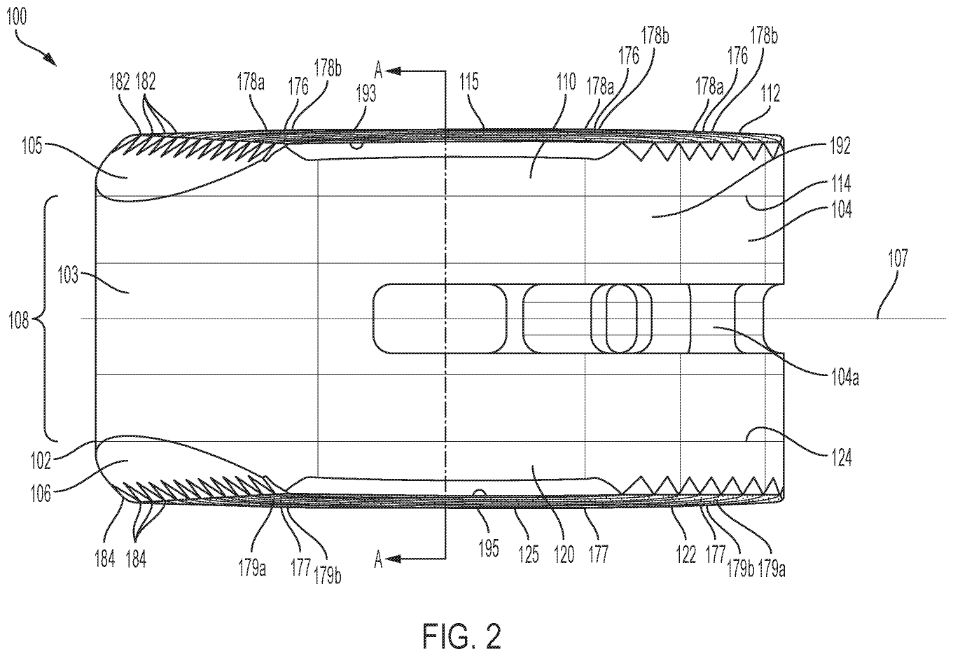

Referring to FIGS. 2 and 4, transverse pins 170-172 each have a first end 176 and a second end 177 opposite the first end. Each first end 176 is positioned flush with first fusion surface 112 of first plank 110. Similarly, each second end 177 is positioned flush with second fusion surface 122 of second plank 120. In this arrangement, first and second ends 176, 177 are positioned to engage cancellous bone and play a role in guiding the insertion of graft 100, in the same manner as first and second fusion surfaces 112, 122. First ends 176 of transverse pins 170-172 each have a third platform 178a and a plurality of third projections 178b raised above the third platform in a third pattern. Second ends 177 of transverse pins 170-172 each have a fourth platform 179a and a plurality of fourth projections 179b raised above the fourth platform in a fourth pattern. Third projections 178b and fourth projections 179b follow the same curvatures and have the same spacings and dimensions as first projections 115 and second projections 125, respectively. Thus, the first pattern of first projections 115 blends with the third pattern of third projections 178b to form a first continuous pattern of curved projections on graft 100. Likewise, the second pattern of second projections 125 blends with the fourth pattern of fourth projections 179b to form a second continuous pattern of curved projections on graft 100. In this arrangement, the ends of transverse pins 170-172 function as fusion surfaces that work in harmony with first and second fusion surfaces 112, 122.

First projections 115 and third projections 178b collectively form a first series of continuous longitudinal ridges 182 extending in parallel. Second projections 125 and fourth projections 179b collectively form a second series of continuous longitudinal ridges 184 extending in parallel. Longitudinal ridges 182, 184 extend parallel to one another and to the curved shape of first plank 110 and second plank 120, respectively.

Body 101 has a first curved side 192 extending between first fusion surface 112 and second fusion surface 122. Body 101 also has a second curved side 194 extending between the first fusion surface 112 and second fusion surface 122 opposite first curved side 192. First curved side 192 intersects first platform 113 along a first curved edge 193. Second curved side 194 intersects second platform 123 along a second curved edge 195. First curved edge 193 and second curved edge 195 are defined by curves that are parallel to longitudinal ridges 182 and longitudinal ridges 184. As such, first curved edge 193, second curved edge 195, and longitudinal ridges 182, 184 are defined by parallel curves.

Longitudinal ridges 182, 184 extend along the full lengths of first and second planks 110, 120, following continuous parallel curves. This curved pattern is advantageous because the longitudinal ridges 182, 184 extend transversely to the trabecular structure of the adjoining vertebrae when graft 100 is in place. The trabecular structure refers to a core of cancellous bone that consists of spicules of bone known as trabeculae. Trabeculae are generally oriented in directions parallel to lines of stress, forming an architecture that resembles a matrix. This matrix bears against graft implants under axial compression. To maximize the support of this load, the curved longitudinal ridges 182, 184 run transversely to the trabecular lines that form the matrix, such that the ridges cross over the trabeculae at multiple intersections, as opposed to running parallel to or between the trabeculae. Increasing the number of intersections between the ridges and the matrix reduces subsidence because the longitudinal ridges support the adjacent vertebrae at multiple points where they intersect with the trabeculae. Therefore, curved longitudinal ridges 182, 184 are superior to spikes or other projections that are arranged in anterior-posterior patterns, because those patterns cross the trabeculae at fewer intersections.

Transverse connectors according to the present disclosure can be formed of synthetic material, allogenic bone, or autogenous bone. In addition, transverse connectors and transverse passages according to the present disclosure can have a variety of cross sectional shapes, including polygonal shapes (regular or irregular polygons), circular, oval, elliptical, or customized shapes. Moreover, transverse pins and transverse passages can all have the same shapes and dimensions, or have different shapes and dimensions from one another. In the present example, transverse pins 170-172 are all formed of cortical bone and have uniform circular cross-sections forming cylinders having the same diameter. Transverse passages 173-175 also have uniform circular cross sections having the same diameter. The diameter of transverse passages 173-175 is substantially equal to the diameter of transverse pins 170-172. It will be appreciated that the cross sectional shapes of transverse pins according to the present disclosure need not have the same shape as their corresponding transverse passages, but can also have different shapes that permit press fitting of the pins into the passages. For example, a graft according to the present disclosure may feature a transverse pin with a hexagonal cross section press fitted into a cylindrical through-bore. Therefore, transverse connectors and passages can feature various combinations of cross sectional shapes.

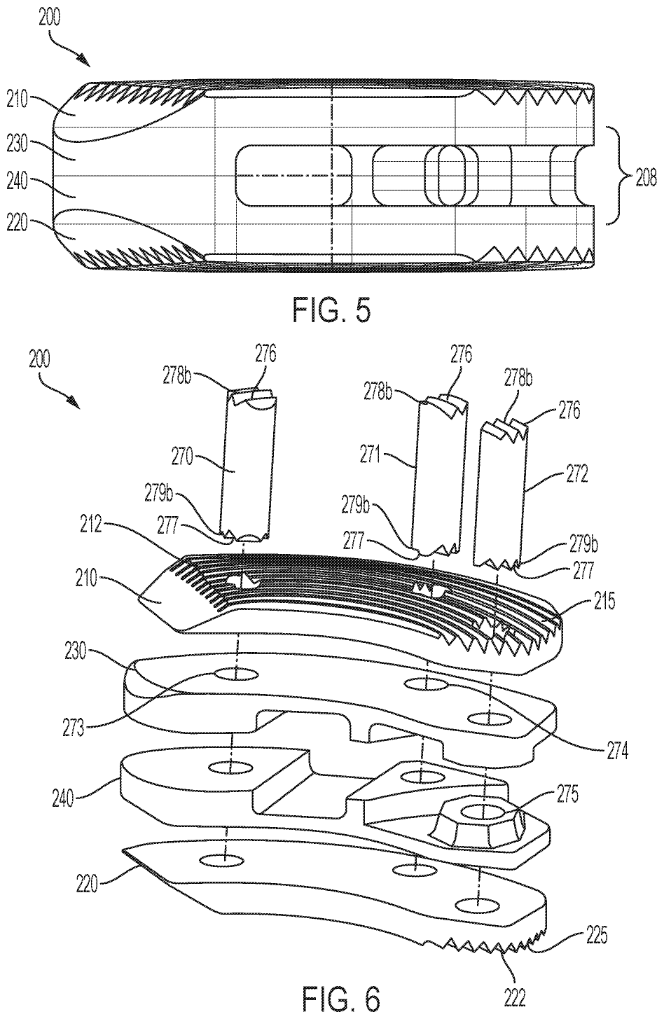

Grafts according to the present disclosure can have any number of planks to accommodate the disc space and maintain a desired spacing. FIG. 5 shows a graft 200 according to another example that is similar to graft 100, but features a different number of planks in the interior section. Graft 200 features a first plank 210, a second plank 220 and an interior section 208 consisting only of a third plank 230 and a fourth plank 240. This version, which has a total of only four planks, can be appropriate for narrower disc spaces that cannot accommodate the full height of a graft having more than four planks.

As with graft 100, the planks of graft 200 are secured together with three transverse pins 270-272. Each transverse pin 270-272 is press fit into a transverse through-bore 273-275, respectively. Transverse pins 270-272 each have a first end 276 and a second end 277 opposite the first end. Each first end 276 is positioned flush with a first fusion surface 212 of first plank 210. Similarly, each second end 277 is positioned flush with a second fusion surface 222 of second plank 220. Transverse pins 270-272 each have projections 278b that blend with projections 215 on first fusion surface 212, and projections 279b that blend with projections 225 on second fusion surface 222.

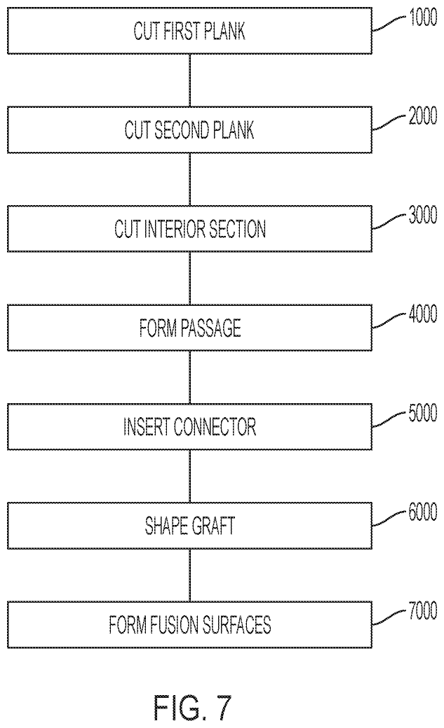

Referring to FIG. 7, a method of manufacturing a graft according to the present disclosure is illustrated. This method can be used to form grafts having one or more features of graft 100, graft 200 or a different graft.

In step 1000, a first plank is cut from allograft material. In step 2000, a second plank is cut from allograft material. The first and second planks are formed with mating surfaces that will mate with other planks. After the mating surfaces are formed, the remaining shape of each plank can be cut in a later step, for example after all of the planks are assembled together. Alternatively, the first and second planks can have some of their outer shape formed during this step. The first and second planks can be formed as mirror images of one another, as done with first and second planks 110, 120, 210, 220 of grafts 100 and 200, or have other configurations.

In step 3000, an interior section is cut from allograft material. This step may include the cutting of a single plank to be placed between the first and second planks produced in the previous steps. Alternatively, this step may include the cutting of two or more planks to be placed between the first and second planks produced in the previous steps. Interior section can be formed as a simple rectangular piece or pieces for shaping in a subsequent step, or have some of its outer shape formed during this step.

In step 4000, at least one transverse passage is formed through the first plank, second plank, and interior section. The transverse passage can be formed by individually drilling a section of the passage in each plank and interior section. Alternatively, the transverse passage can be formed by arranging the first plank, interior section, and second plank in a vertically stacked arrangement, and then drilling the passage in the planks and interior section in a single operation. In either approach, drilling can be done in a manner that forms a passage through all layers, for example by drilling one or more through-bores. Alternatively, multiple passages can be drilled through less than all layers, so long as every layer has at least one section of one passage for receiving a transverse connector.

In step 5000, at least one transverse connector is inserted into the at least one transverse passage to interconnect the first plank, interior section and second plank. This can be done by stacking the first plank, interior section and second plank so that their passage sections are coaxially aligned. Once the passage sections are aligned, the at least one transverse connector can be inserted into the passage sections by press fitting or other suitable method.

In step 6000, the first plank, interior section and second plank are machined or otherwise processed as an assembly to receive their final shaping, to the extent that shaping has not already been completed. This may include machining to form the leading and trailing ends, the instrument engagement section, or any other aspect of the implant's geometry. The finished shape can be any desired geometry, such as the kidney bean shape of graft 100 or graft 200, or other shape.

In step 7000, fusion surfaces are formed in the first plank and second plank, for example by milling. A fusion surface is also milled into one or both ends of the at least one transverse connector, in cases where the end or ends are flush with outer surfaces of the first and second planks. Milling is done to form a continuous pattern of projections that extend over the outer surfaces of the planks, and if applicable, the ends of the transverse connector(s). The patterns of projections on each side of the graft can be configured to influence and guide the direction of advancement of the graft. For example, the projections can be formed as arc-shaped ridges running continuously along the top of the first plank and bottom of the second plank, as done with grafts 100 and 200.

The steps described above can be performed in the order shown in FIG. 7, or performed in a different order. In addition, some steps described above, like shaping, need not be completed prior to moving to a subsequent step. Rather, some steps can be partially completed before moving to a subsequent step, and then resumed after the subsequent step is completed. Moreover, some steps described above can occur simultaneously, and need not be done sequentially.

Although this description makes reference to specific embodiments and illustrations, the present disclosure is not intended to be limited to the details shown. Rather, the present disclosure encompasses various modifications and combinations of embodiments, features and steps described herein, as well as other variations that may be made within the scope and range of the claims and equivalents.

Accordingly, it is intended that the appended claims cover all such variations as fall within the scope of the present disclosure.

1. An allograft implant comprising:
a first plank formed of allograft, the first plank comprising a first fusion surface and a first mating surface opposite the first fusion surface;
a second plank formed of allograft, the second plank comprising a second fusion surface and a second mating surface opposite the second fusion surface;
at least one interior plank formed of allograft, the at least one interior plank comprising a third mating surface attached to the first mating surface of the first plank, and a fourth mating surface opposite the third mating surface and attached to the second mating surface; and
at least one transverse connector interconnecting the first plank, second plank and the at least one interior plank.

2. The allograft implant of claim 1, wherein the first plank, second plank and the at least one interior plank define at least one transverse passage, the at least one transverse passage extending through at least a portion of the first plank, at least a portion of the second plank, and at least a portion of the at least one interior plank.

3. The allograft implant of claim 1, wherein the at least one transverse passage comprises at least one through-passage that extends from the first fusion surface of the first plank to the second fusion surface of the second plank.

4. The allograft implant of claim 1, wherein the at least one transverse connector interconnects the first plank, second plank and the at least one interior plank through the at least one transverse passage.

5. The allograft implant of claim 1, claims, wherein the at least one transverse connector is press fit through the at least one transverse passage.

6. The allograft implant of claim 1, wherein the at least one transverse connector comprises a first end and a second end opposite the first end.

7. The allograft implant of claim 6, wherein the first end is positioned flush with the first fusion surface of the first plank, and the second end is positioned flush with the second fusion surface of the second plank.

8. The allograft implant of claim 1,
wherein the first fusion surface comprises a first platform and a plurality of first projections raised above the first platform in a first pattern, and
wherein the second fusion surface comprises a second platform and a plurality of second projections raised above the second platform in a second pattern.

9. The allograft implant of claim 8,
wherein the first end of the at least one transverse connector comprises a third platform and a plurality of third projections raised above the third platform in a third pattern, and
wherein the second end of the at least one transverse connector comprises a fourth platform and a plurality of fourth projections raised above the fourth platform in a fourth pattern.

10. The allograft implant of claim 9,
wherein the first pattern of first projections blends with the third pattern of third projections to form a first continuous pattern of projections, and
wherein the second pattern of second projections blends with the fourth pattern of fourth projections to form a second continuous pattern of projections.

11. The allograft implant of claim 8,
wherein the first projections and third projections comprise a first series of longitudinal ridges extending in parallel, and
wherein the second projections and fourth projections comprise a second series of longitudinal ridges extending in parallel.

12. The allograft implant of claim 11, wherein the first plank and the second plank are arc-shaped.

13. The allograft implant of claim 12, further comprising a first curved side extending between the first fusion surface and the second fusion surface, and a second curved side extending between the first fusion surface and the second fusion surface opposite the first curved side.

14. The allograft implant of claim 13, wherein first curved side intersects the first platform along a first curved edge, and the second curved side intersects the second platform along a second curved edge.

15. The allograft implant of claim 14, wherein the first curved edge and second curved edge are defined by parallel curves.

16. The allograft implant of claim 15, wherein the first curved edge, second curved edge, and first series of longitudinal ridges are defined by parallel curves.

17. The allograft implant of claim 1, wherein the at least one transverse connector is formed of allograft.

18. The allograft implant of claim 1, wherein the at least one transverse connector comprises a first transverse pin and a second transverse pin extending parallel to the first transverse pin.

19. The allograft implant of claim 1, wherein the at least one transverse connector and the at least one transverse passage are cylindrical.

20. A method of manufacturing an allograft implant comprising the steps of:
cutting a first plank from allograft material;
cutting a second plank from allograft material;
cutting at least one interior plank from allograft material;
drilling a first transverse passage through the first plank, a second transverse passage through the second plank, and a third transverse passage through the at least one interior plank;
stacking the first plank, second plank and at least one interior plank with the first transverse passage, second transverse passage and third transverse passage coaxially aligned; and
press fitting a transverse connector through the first transverse passage, second transverse passage and third transverse passage to interconnect the first plank, second plank and at least one interior plank.
